(19) United States
(12) Patent Application Publication
(10) Pub. No.: US 2017/0269053 A1
(43) Pub. Date: Sep. 21, 2017

(54) SYSTEM AND METHOD FOR PROCESSING PETROLEUM

(71) Applicant: William ARDREY, Cleveland, OK (US)

(72) Inventors: William E. Ardrey, Cleveland, OK (US); Alexander Harris Myers, Tulsa, OK (US)

(21) Appl. No.: 15/529,259

(22) PCT Filed: Dec. 7, 2015
(86) PCT No.: PCT/US2015/064327

§ 371 Date: May 24, 2017

Related U.S. Application Data
(60) Provisional application No. 62/089,079, filed on Dec. 8, 2014.

(51) Int. Cl. G01N 33/28; G05B 15/02; G01F 23/00

(52) U.S. Cl. CPC G01N 33/28; G01F 23/0007; G05B 15/02; G06Q 50/06

(57) ABSTRACT

A system and method for monitoring and treating petroleum in a storage tank is provided. The system and method comprise including a plurality of vertically spaced sensors inside the tank, the plurality of vertically spaced sensors being in communication with a computer. The computer is able to calculate the quantity and quality of the petroleum in the tank based off the readings from the plurality of vertically spaced sensors. As a result, the petroleum in the tank can be optionally treated prior to sale in order to optimize the price received for the petroleum in the tank.

### CROSS REFERENCE TO RELATED APPLICATION

This application claims the benefit of U.S. Provisional Application 62/089,079 filed on Dec. 8, 2014, which is incorporated by reference.

### FIELD

The present disclosure relates to the storing and distribution of petroleum and, more particularly, to measuring and tracking the quantity and quality of petroleum that is stored or distributed.

### BACKGROUND

Petroleum is often transferred between buyers and sellers. For example, producers of petroleum sell petroleum to refiners who process and market refined petroleum products. Additionally, petroleum can be transferred or sold to one or more intermediate parties before being sold to a refiner. Between transfers, petroleum is sometimes contained in storage tanks that allow the seller to store the petroleum while waiting for the buyer to take custody of the petroleum. These storage tanks can be above ground storage tanks, below ground storage tanks, tanker cars on trains, tanker trucks and the like.

The value or price of the petroleum depends on a variety of factors. Such factors include the formation from which the petroleum was produced, the sulfur content of the petroleum, the density or API gravity of the petroleum and the water content of the petroleum. Generally, the lower the density of the petroleum, the higher the price of the petroleum. Light petroleum will produce a higher yield of gasoline or diesel when it is refined than heavy petroleum. Similarly, the lower the water content of the petroleum, the higher the price received because water is an impurity that typically needs to be removed from the petroleum.

To determine the value or price of petroleum, sellers sometimes process their petroleum through a lease automatic custody transfer (“LACT”) unit. Essentially, a LACT unit is a piece of equipment which measures the quality and quantity of a petroleum product as it is transferred from the custody of the seller to the buyer. The price of the petroleum is determined by the LACT unit's measurements of the petroleum's attributes. The LACT unit can be contained in a truck or can be part of a pipeline that receives petroleum.

While a LACT unit can accurately measure the quantity and quality of petroleum, it only does so when petroleum is transferred from the seller to the buyer. Consequently, the producer has little choice other than to sell their petroleum to buyers when their tanks are nearly full and accept whatever price the measurements made by the LACT unit dictate. Accordingly, the seller has little ability to optimize the condition of the petroleum, maximize the price received for the petroleum or negotiate the price for which the petroleum is sold.

### SUMMARY

In one embodiment, the present disclosure relates to a system for determining the quality and quantity of petroleum. The system can include a tank having a height, the tank suitable for storing the petroleum and containing the petroleum. The system can further include a plurality of sensors spaced apart along the height of the tank, such that there is a sensor height associated with each sensor. Each sensor is configured to measure one or more attributes of a portion of the petroleum located at the associated sensor height. The system can further include a computer processor or a plurality of processors configured to receive the measured attributes from the sensors and calculate the quality of the portion of the petroleum at each sensor height.

In another embodiment, the system sensors can measure a dielectric constant, conductivity, temperature and pressure.

In another embodiment, the system computer processor calculates one or more calculated values from the attributes and determines the quality of the petroleum from the calculated values. The calculated values of the petroleum can include water content, salt content, basic sediment and water (“BS&W”) content, API gravity and free water content.

In some embodiments, the system further comprises a transmission unit, wherein the transmission unit is in electronic communication with the sensors and the computer processor. The system can also comprise a petroleum output, a treatment unit and a system output. The petroleum output can be associated with the tank such that petroleum can be removed from the tank. The treatment unit can be in fluid flow communication with the petroleum output such that the treatment unit can be activated to receive petroleum from the tank and treat the petroleum to remove one or more components from the petroleum to produce treated petroleum. The system output can be in fluid flow communication with the petroleum output and the treatment unit such that at least a portion of the petroleum removed from the tank can be removed from the system without treatment and such that at least a portion of the treated petroleum can be removed from the system.

Additionally, the computer can be configured to determine an advantageous disposal of the petroleum based on the quality of the petroleum at each sensor height. The advantageous disposal can includes selling the first portion of the petroleum removed from the tank upon a first set of conditions, selling the portion of the treated petroleum upon a second set of conditions, and selling no portion of the petroleum upon a third set of conditions. Also, the computer can be configured to direct the petroleum output to introduce the first portion of petroleum to the system output upon the first set of conditions, and direct the petroleum output to introduce some or all of the petroleum removed from the tank to the treatment unit and activating the treatment unit upon the second set of conditions.

In some embodiments, the system further comprises an agitator associated with the tank such that when activated the agitator blends the petroleum in the tank to produce a homogenated petroleum. The computer can be further configured to activate the agitator upon a fourth set of conditions.

In other embodiments, the present disclosure relates to a method for determining quality of petroleum in a tank. The method comprising:
(a) measuring one or more attributes of the petroleum, wherein the attributes are measured at a plurality of heights within the tank such that a set of attributes is measured for each height, and the set of attributes reflect the properties of the petroleum at the height at which the set was taken;
(b) transmitting each set to a computer;
(c) calculating from each set one or more calculated values, which reflect the quality of the petroleum at the corresponding height;
(d) determining an advantageous disposal of the petroleum based on the quality of the petroleum at each height; and
(e) processing the petroleum based on the advantageous disposal.

In the method, the attributes of the petroleum can include a dielectric constant, conductivity, temperature. In some embodiments, the attributes will also include pressure as a function of height. Also in the method, the calculated values can include suspended water content, salt content, BS&W content, API gravity and free water content.

In some embodiments of the method, the advantageous disposal includes the options of selling an untreated portion of the petroleum upon a first set of conditions; selling a treated portion of the petroleum upon a second set of conditions; and selling no portion of the petroleum upon a third set of conditions, The treated portion has been treated to remove one or more components from a portion of petroleum withdrawn from the tank, and the untreated portion of petroleum has no components removed from the portion of petroleum withdrawn from the tank.

In some embodiments, the step of determining an advantageous disposal includes determining the profit associated with options. Also, the advantageous disposal further includes the option of blending the petroleum in the tank to create a homogenous petroleum prior to selling the untreated portion and the option of not blending the petroleum in the tank prior to selling the untreated portion.

Additionally, the advantageous disposal can include the option of blending the petroleum in the tank to create the homogenous petroleum prior to treating the portion of petroleum withdrawn from the tank, and the option of not blending the petroleum in the tank prior to treating the portion of petroleum withdrawn from the tank.

### BRIEF DESCRIPTION OF THE FIGURES

FIG. 1 is a schematic illustration of one embodiment of a monitoring system described by the present application.

FIG. 2 is a schematic illustration of a petroleum distribution system having a tank and monitoring system in accordance with some embodiments.

FIG. 3 is an illustration of the relationship between measured attributes and calculated values used to determine the calculated values from the measured attributes.

FIG. 4 is a flow chart illustrating analysis related to determining the disposition of the petroleum based on the measurements taken.

FIG. 5 is a schematic illustration of petroleum distribution system having multiple tanks and a monitoring system in accordance with some embodiments.

### DETAILED DESCRIPTION

Referring now to the drawings, wherein like reference numbers are used to designate like elements throughout the various views, various embodiments are illustrated and described. The figures are not necessarily drawn to scale, and in some instances the drawings have been exaggerated or simplified in places for illustrative purposes. Where components of relatively well-known designs are employed, their structure and operation will not be described in detail. One having skill in the art will appreciate the many possible applications and variations of the present disclosure based on the following description.

As used herein, petroleum means a composition comprising hydrocarbons. Petroleum can exist as a liquid, solution or as a multi-phase system. In some cases, petroleum contains impurities such as solids produced with the petroleum from a subterranean formation, water produced with the petroleum from the formation, or chemical impurities like sulfur or hydrogen sulfide. In many situations, petroleum can be produced from the ground and deposited in tanks.

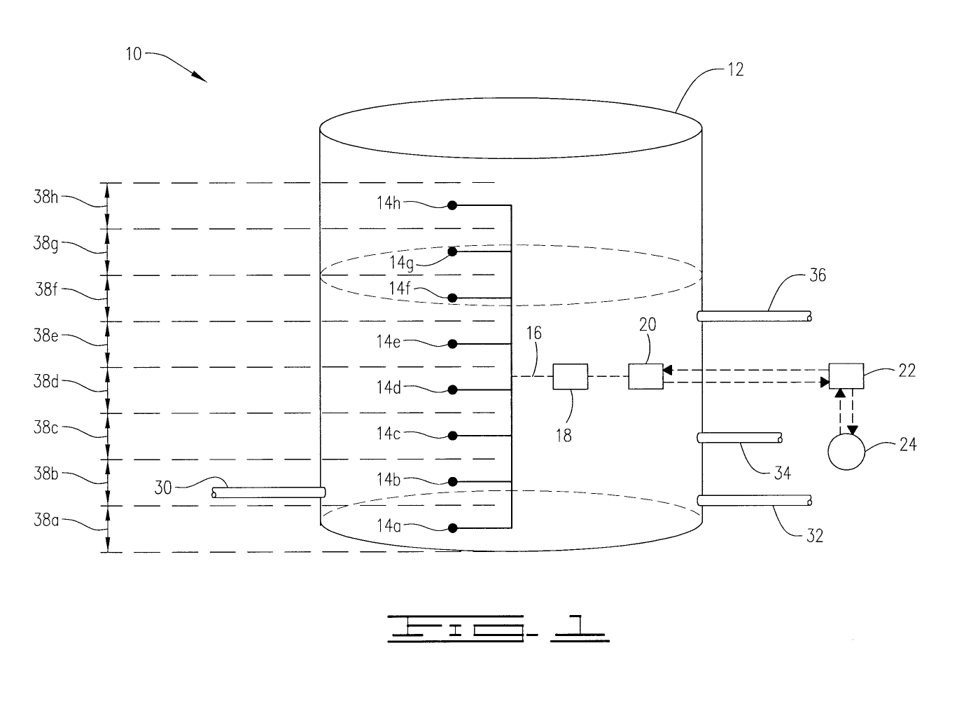

Referring to FIG. 1, a system 10 for measuring the quality and quantity of petroleum inside a tank 12 is illustrated. The tank 12 contains a plurality of sensors 14. The plurality of sensors 14a-h is made up of individual sensors 14a through 14h each at a different height in the tank 12. Each sensor 14 is capable of measuring an attribute of petroleum contained in the tank 12. As used herein, an attribute of petroleum refers to a property of the petroleum that can be measured. For example, temperature, dielectric constant, conductivity and hydraulic pressure are all attributes of petroleum. The dielectric constant is also sometimes called the relative permittivity. Additionally, in some applications, each sensor can contain an accelerometer to determine orientation of the sensors. The accelerometer can be used to calibrate alignment issues (correct the height between sensors), as well providing an alert if the sensors appear to come loose or otherwise find themselves grossly out of alignment.

In some embodiments, each sensor can measure one or more attributes selected from the group consisting of temperature, dielectric constant, conductivity and the hydraulic pressure of the petroleum. While FIG. 1 shows one individual sensor at each height, one skilled in the art will understand by examining this disclosure that that multiple sensors can be used at each height and that each sensor can measure one or more attributes of the tank.

The sensors 14a-h within the tank 12 can communicate using a communication apparatus 16 with a computer 18. The communication apparatus 16 will typically use electronic communication. For example, a wired electronic communication system can be used. However, the communication apparatus 18 can also use infrared, radio, hydraulic or other suitable communication mechanisms.

The computer 18 should be configured to receive information from the sensors 14 and make calculations based on the data received from the sensors 14a-h. The computer 18 can comprise memory and a processor. Optionally, the computer 18 can communicate with a transmission unit 20. As used herein, a transmission unit 20 is an electronic device capable of sending and receiving electronic information. The transmission unit can communicate with a computer network 22 which can communicate with a central database 24. The tank 12 is also configured to receive petroleum from petroleum input 30. The petroleum input 30 can receive petroleum from a well, pipeline, another tank, a truck or any other device suitable for storing or transporting petroleum.

Additionally, petroleum can be removed from tank 12 through one or more petroleum outputs. Generally, there will be at least a first petroleum output 32, typically located at or below zone 38a. In some embodiments, there will be two or more such petroleum outputs in order that portions of the tank contents can be removed from different zones 38a-h within tank 12. Illustrated in FIG. 1 is first petroleum output 32, which is at the bottom of tank 12, second petroleum output 34, which is above first petroleum output 32 but below the mid-height of tank 12, and third petroleum output 36, which is higher than petroleum output 34 but below the top-most zone 38h. Petroleum outputs 32, 34 and 36 can transfer petroleum from tank 12 to a pipeline or other transportation unit. In some embodiments, petroleum outputs 32, 34 and 36 can transfer the petroleum to another operational unit. For example, the petroleum can be transferred to a separation process, or heat treatment device to remove the water content, or to a hydrocyclone or centrifuge separators to remove BS&W. Any other process useful for treating petroleum can also be used in connection with the disclosed system and method.

Petroleum outputs 32, 34 and 36 can be used to sell portions of the petroleum having a higher content of lower density hydrocarbons and lower water content separately from portions of petroleum having a lower content of lower density hydrocarbons and higher water content. This is because lower density hydrocarbons (typically, lower molecular weight hydrocarbons) will have a tendency to rise in tank 12 whereas higher density hydrocarbons (typically, higher molecular weight hydrocarbons) and water will have a tendency to descend in tank 12.

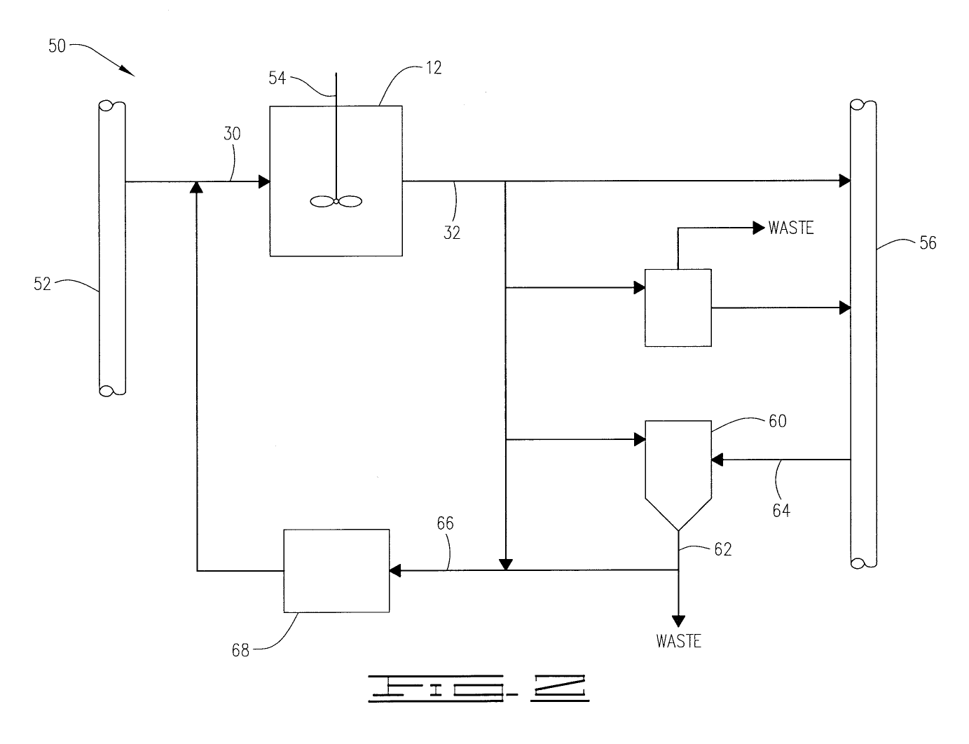

Turning to FIG. 2, a petroleum storage and distribution system 50 is shown. Petroleum is received by tank 12 from a petroleum transportation device (shown as pipeline 52) through petroleum input 30. During storage in tank 12, components of the petroleum can separate. Accordingly, tank 12 can include an agitator 54 that can be used to mix or blend the petroleum prior to removal from tank 12.

In operation, petroleum is added to the tank 12 as it is received. The petroleum will generally have been produced from a well. Alternatively, the petroleum can come directly or indirectly from a petroleum producer.

As outlined below, the quality and quantity measuring system 10 can used to determine how and when the petroleum should be sold. Accordingly, once the determination is made, petroleum can be removed from tank 12 through petroleum output 32 and transferred directly to a transportation device, such as pipeline 56, a tanker railway car or tanker truck. Alternatively, the petroleum removed from tank 12 can be introduced into a treatment unit to enhance its value prior to introduction into the transportation device. For example, the petroleum can be introduced into a treatment unit, such as a heat treater device 58 or separation unit 60, for separating out water, BS&W or for separating out heavy and lighter components of the petroleum for separate sale or distribution. Separation unit 60 can be any suitable separation device such as a hydrocyclone, centrifuge separators or distillation tower. After treatment, a first portion 62 from treatment unit 60 is returned to tank 12 or taken off as waste. A second portion 64 is introduced to pipeline 56 or another petroleum transportation unit.

Additionally, a portion of petroleum from output 32 can be returned to tank 12 through line 66, which may include a holding tank 68. In this manner, another system for selling petroleum from different zones 38 of tank 12 is provided. For example, a portion of the petroleum from some of zones 38 can be sent to the pipeline 56 or a treatment unit, while another portion of petroleum from other zones 38 is returned to tank 12. If necessary, the portion to be returned to tank 12 can be held in tank 68 while the other portion of petroleum is being removed so as to prevent mixing of the returning portion into the portion to be sent to pipeline 56. In this manner, portions of the petroleum having a higher content of lower density hydrocarbons and lower water content can be sold separately from portions of petroleum having a lower content of lower density hydrocarbons and higher water content. Further, portions returned to tank 12 can be allowed additional time for stratification to occur thus allowing for further separation of lower density hydrocarbons from higher density hydrocarbons and water.

To sell or transfer the petroleum or a portion thereof, the quality and quantity of the petroleum will usually need to be determined. Information on the quality and quantity of the petroleum is derived from readings from the plurality of sensors 14. The plurality of sensors 14 measure attributes of the petroleum. As indicated, the plurality of sensors 14 is arrayed along the height of the tank 12. In this way, the sensors 14 account for variations in temperature, pressure and the like along the height of the tank 12. From the data obtained by the sensors, the overall quantity and quality of petroleum in the tank 12 can be determined despite such variations along the height of the tank.

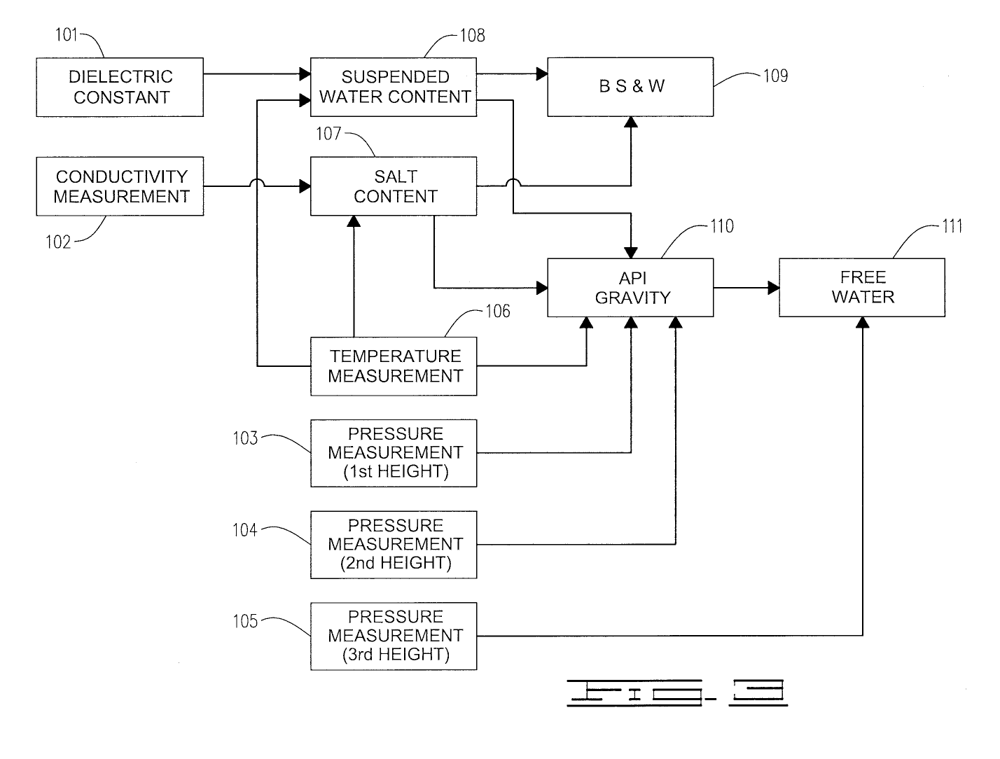

FIG. 3 illustrates the relationship between measured attributes and calculated values that computer 18 or computer network 22 uses to determine the calculated values from the measured attributes. As used herein, a calculated value is a characteristic of the petroleum that requires calculation, typically a calculation involving an attribute of the petroleum. For example, calculated values include suspended water content, salt content, BS&W content, API gravity and free water content. Additionally, hereinafter when the disclosure mentions computer 18 or computer network 22, it will be understood that the function described can be performed by either computer network 22 or computer 18.

Sensors 14a-h measure various attributes of the petroleum at each height in the tank 12. Typically, the attributes measured can include at least one attribute selected from the group consisting of dielectric constant 101, conductivity 102, temperature 106, and pressure 103, 104 and 105. More typically, the attributes can include measurements for dielectric constant 101, conductivity 102, temperature 106, and pressure at each height. For example, as illustrated in FIG. 3, pressure can be taken from a first height 103, a second height 104 and third height 105. The first height can be at the bottom of the tank, the third height near the top of the petroleum in the tank, and the second height at a position between the first height and third height. It will be understood that although FIG. 3 only shows a single measurement for each of dielectric constant 101, conductivity 102 and temperature 106 these also can include measurements of these attributes from sensors at different height. The different height measurements are not shown in FIG. 3 for simplicity of the figure. By measuring the attributes at each height, there is generated a set of attributes for each height, which reflects the properties of the petroleum located at the height at which the set was taken.

From these attributes, various calculated values representing characteristics of the petroleum can be calculated. These calculated values can typically include at least one calculated value selected from the group consisting of suspended water content 108, salt content 107, basic sediment and water (“BS&W”) content 109, API gravity 110 and free water content 111. The calculated values can include calculated values for suspended water content 108, salt content 107, BS&W content 109, API gravity 110 and free water content 111 at each height. For example, the temperature of the petroleum at each height in the tank 12 and the dielectric constant of the petroleum at each height can be measured. From this data, the suspended water content of the petroleum can be calculated 108 by the computer 18 using the measured temperature 106 and dielectric constant 101 at each height to calculate the suspended water content 108 of the petroleum at each height. Additionally, the overall suspended water content of all the petroleum in the tank can be calculated.

Similarly, sensors 14a-h measure the conductivity 102 of the petroleum at each height to calculate the salt content 107 of the petroleum. From the suspended water and salt content, the computer 12 can calculate the BS&W content of the petroleum 109. The BS&W in petroleum has little or no economic value. Therefore, it often is desirable for a producer to know the BS&W content of the tank in order to better understand the total value of the petroleum stored in the tank. BS&W refers to water and solids contained in petroleum and the solids can also be referred to as sediment or mud.

From the suspended water content, the salt content, the temperature measurements and a multitude of pressure measurements, computer 18 can calculate the overall API gravity of the petroleum in the tank 110. As used herein, the API gravity is the measurement used by the American Petroleum Institute to measure how heavy or light (dense) petroleum is compared to water. The API gravity can be calculated from the relative density of petroleum to water by the formula:

$$API gravity = \frac{141.5}{R D} - 131.5$$

The relative density (RD) of petroleum is the density of the petroleum divided by the density of water under similar conditions. As will be understood by one skilled in the art, calculating the API generally will require measuring at least two pressure readings at different heights so that the API gravity can be determined from the delta of two density measurements.

Once the API gravity is obtained, sensor 14a provides pressure measurements 103 from the bottom of the tank, other sensors 14b-g may provide pressure from one or more intermediate points in the tank, and sensor 14h provides pressure from the top of the tank 3. The API gravity measurement is used along with the pressure measurement to calculate the free water content of the tank 3.

Further, from the measured attributes, the computer can calculate the overall quantity of petroleum within the tank and can determine the quantity of petroleum which has certain predefined calculated values. For example, the computer can calculate the quantity of petroleum having a BS&W content with in a range, such as from 0.5% to 0.7%, and which zones 34a-h have that BS&W content.

All of the above data, especially the BS&W, API gravity and free water content of the petroleum, is used to determine how and when to sell the petroleum contained in the tank 12. From the above information, the system determines how to maximize the profit obtained from the petroleum in the tank.

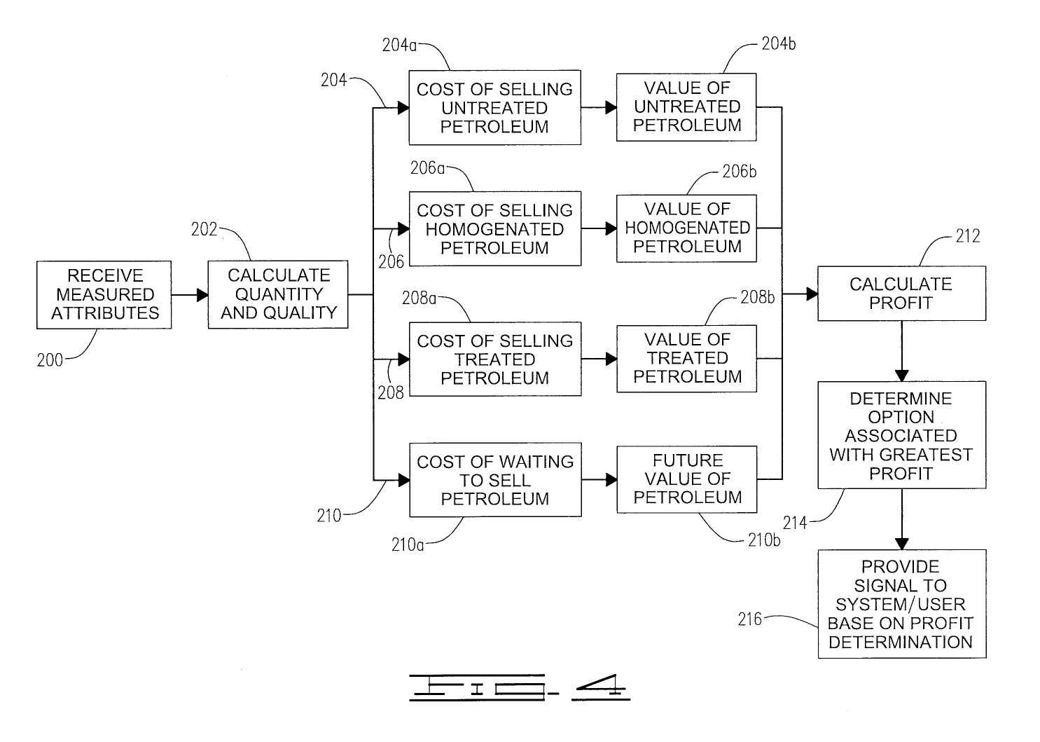

Turning to FIG. 4, the process of determining how to maximize the profit obtained from the contents of tank 12 is illustrated. First, at step 200 the computer network 22 or a computer 18 receives the measured attributes from the sensors 14. As indicated above, when the disclosure mentions computer 18 or a computer network 22, it will be understood that the function described can be performed by either computer network 22 or computer 18.

From the measured attributes, computer 18 calculates the quality and quantity of the petroleum in tank 12 in step 202. For example, computer 18 calculates the water and BS&W contents of tank 12. This can include computer 18 calculating quality and quantity of the contents of the tank at various heights within tank 12. That is, determining the quality and quantity of specific portions of the tank represented by a zone (shown as zone 38a-h in FIG. 1) around a specific tank height in which the sensors associated with that height take measurements. Also, this can include computer 18 determining the quality and quantity of the tank as a whole. That is, what the quality and quantity of the contents will be if blended together as a homogenous whole.

Afterwards, the computer 18 estimates the cost and value associated with a variety of options in treating the contents of tank 12. For example, the owner or distributer may have a first option 201 of selling all or a portion of the petroleum untreated 201, a second option 202 of homogenizing the contents of tank 12 before selling the petroleum, a third option 203 of treating all or a portion of the petroleum for sale, a fourth option 204 of waiting to sell the contents of tank 12 to a later date.

Thus, computer 18 estimates the costs associated with the variety of methods of treating the contents of tank 12. For example, the computer 18 can calculate the cost of selling the contents of tank 12 without any treatment (step 204a), of homogenizing the contents of the tank and selling them (step 206a), of removing and treating all or a portion of the tank (step 208a) or waiting to sell the contents of the tank (step 210a). The present disclosure can also include any other unit operation suitable for treating petroleum. Suitable operations typically have relatively predictable, calculable costs.

Also, computer 18 can estimate the likely value of the tank contents based on the various treatment options. For example, the system calculates the value of the untreated tank contents (step 204b), the value of the homogenized tank (step 206b), the value of the tank contents with portions treated (step 208b), and the estimated value of the contents of the tank in the future (step 210b). In step 212, computer 18 calculates the expected profit from each possible course of action based on subtracting the expected costs of treatment from the expected value realized from the contents of tank 12, typically using commodities pricing data. Subsequently, computer 18 determines which option is likely to generate the greatest profit in step 214. Finally, in step 216, computer 18 provides a signal to the system, which results in the system performing the tasks necessary to achieve the highest profit. For example, if the system calculates that homogenizing the tank and selling the homogenized contents will realize the greatest profit, the system will transmit a signal that activates agitator 54 or another suitable device associated with tank 12, to homogenize or blend the contents of the tank at step 216. The system can calculate the likely price of the contents of the tank using a variety of methods. For example, the system can use the local spot price of petroleum or any other method for calculating the likely price of petroleum. For example, the value of petroleum is available on line in real time. The individual steps described in FIG. 4 are more fully described below.

In one exemplary embodiment, the system 10 determines the cost of selling the petroleum in tank 12 without treatment in step 204a. The value of selling the petroleum in tank 12 without treatment is also calculated in step 204b. The cost and value calculations can be made for the total volume of the tank; however, more typically, the cost and value calculations will be made first for each zone 38. Thus, in step 212, profit calculations can be made for selling all the tank contents and/or for selling one or more portions of the tank contents where each portion is associated with at least one of zones 38. The profit calculations constitute a set of conditions on the computer, which can determine an advantageous disposal of the petroleum. Based on these calculations, the computer might determine that the set of conditions indicate it is most advantageous to sell selected portions of the tank content and hold other portions for future sale. For example, if price indicators and future price trends indicate that light petroleum fractions are likely to increase in value in the near future and heavy petroleum fractions are likely to decrease in value, then the system could determine that the higher zones 38e-h contents should be retained and the lower zone contents 38a-d should be sold. In which case, the lower zone contents 38a-d would be drained off through petroleum output 32 and the higher zone contents 38e-h would be retained in tank 12. This represents that, during storage in tank 12, water and the higher density hydrocarbons will tend to settle in the lower zones of the tank, and lower density hydrocarbons will tend to rise to the higher zones of the tank. Thus, the lower zones 38 of tank 12 will tend to have a higher concentration of higher density hydrocarbons and water, while the higher zones 38 will tend to have a higher concentration of lower density hydrocarbons.

Alternatively, the system might determine that the set of conditions indicate that the entire tank should be sold to two or more separate purchasers. For example, the higher zone contents 38a-d could be sold to a first purchaser and the lower zone contents 38e-h could be sold to a second purchaser.

Further, the system might determine that the set of conditions indicate that the entire tank should be sold to one purchaser. Such a sale would involve pumping the total volume of the tank, including the BS&W portion of the tank. The value of the contents of the tank would be based on the total volume of the tank in step 204b.

Additionally, the system determines cost of mixing and homogenizing the tank 206a. For example, the system will account for the cost of mixing the tank using an impeller, pump or the like. In some instances, the value realized from the homogenous tank 205b will be greater for tank contents where the BS&W have been removed. In other instances, a higher value can be obtained by homogenizing the entire petroleum and any BS&W and selling the combined volume.

Further, the system can calculate the costs of removing and or treating all or a portion of the tank contents in step 208a, as well as the expected value realized by doing so in step 208b. Generally, the treatments will be relatively simple separation processes, such as heat treatment to remove water or hydrocyclone separators, centrifuge separators or distillation towers.

For example, the system can calculate the cost removing the portion of tank contents having the highest BS&W content, typically the bottom portion of the tank, in step 208a, as well as the expected value realized by doing so 208b. Thus, the system might determine that the high-BS&W-content portion should be separated from the rest of the tank contents to realize the greatest profit. The system can then remove, by pumping or other similar means, the high-BS&W-content portion of the tank. The high-BS&W-content portion can then be sold separately, disposed of or treated depending on what generates the most favorable profit.

If the high-BS&W-content portion of the tank is not sold, it should be either treated or disposed of Therefore, in order for the high-BS&W-content portion to be removed and treated/disposed of, the extra income generated from selling the tank without high-BS&W-content portion should exceed the cost of treating or disposing of the BS&W portion of the tank. Thus, in step 214, the system can determine the high-BS&W-content portion of the tank should be removed and treated to produce a low-BS&W-content portion by removing water and sediment, and then either sold separately from the rest of the tank contents or returned to the tank to be sold along with the rest of the tank. If returned, the combined volume in the tank may or may not be homogenized. Again, the high-BS&W-content portion will only be treated and returned if the extra price realized from selling the treated bottom portion exceeds the cost of treating the bottom portion.

Finally, the system can also estimate whether the contents of the tank should not be sold and whether the system should wait for the tank to fill further 210. In step 210a, the system calculates the costs associated with waiting to sell the tank including the time value of money. In step 210b, the system calculates the value of waiting to sell the contents. Waiting to sell the contents of the tank could be beneficial if the price of petroleum were expected to rise or the expected future composition of the tank contents would create additional value. Additionally, waiting to sell the contents provides time for greater stratification of the contents; that is, it allows for lower-density and higher-value portions of the contents to separate from higher-density and lower-value portions of the contents and allows for more water to separate from the hydrocarbons. Thus, after additional stratification has occurred, the separated components of the tank might be sold for a greater value than the tank contents would have before the additional stratification.

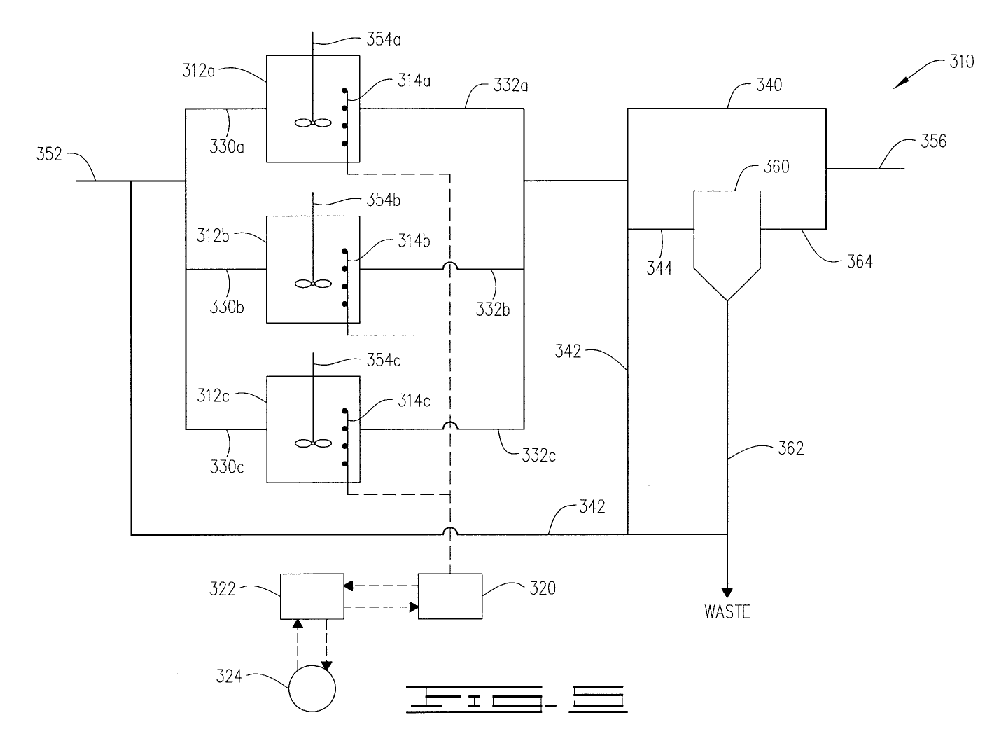

While the above discussion describes only one tank 12, it will be understood by one skilled in the art that the system can have a plurality of such tanks. Thus, with reference to FIG. 5, the present application also relates to a system 310 that integrates a plurality of tanks 312a-c into a network for central automated control of one or more of the plurality of tanks. Each tank 310 can have an agitator 354a-c. System 310 further includes pipeline 352 or other petroleum source, which introduces petroleum into tanks 312a-c through petroleum inputs 330a-c. Petroleum can be removed from tanks 312a-c through petroleum outputs 332a-c. The petroleum removed can be introduced to a pipeline 356, or another petroleum transportation unit, through conduit 340 or can be return to tanks 312a-c through line 342. Alternatively, the petroleum removed can be introduced into a treatment unit 360 through line 344. After treatment, a first portion from treatment unit 360 can flow through conduit 362 to return to tanks 312a-c or taken off as waste. A second portion is introduced through conduit 364 to pipeline 356 or another petroleum transportation unit. Line 340 and line 364 serve as system outputs.

In each individual tank, the plurality of sensors 314a-c will be in communication with a transmission unit 320. As used herein, a transmission unit 320 is an electronic device capable of sending and receiving electronic information. Each transmission unit 320 will send the information produced by the plurality of sensors to the central database 324. The central database 324 will store the most recent measurements produced by the plurality of sensors for each tank. Additionally, the central database 324 can store the history of each measurement for each tank. Further, the central database 324 can store the history of various actions recommended by the computer network 322.

The central database 324 is in communication with the computer network 322 that will assist in any necessary calculations required by the system. In some embodiments, the sensors 314a-c will continuously measure the properties of their associated tank 312a-c and continuously send such information to the central database 324. For example, sensors 314a-c could measure a property of tanks 312a-c every second and the transmission unit 324 could send the information to computer network 322 and central database 324 every second. Computer network 322 can, but need not, send information back to each tank at the same interval.

Based on measurements taken and calculations made, the computer network 322 will then make a determination for how the content of each tank should be disposed of, i.e. selling all or a portion of the content, either with or without treatment; waste disposal of all or a portion of the content, holding all or a portion of the content in one or more of the tanks 312a-c. The determinations can then be executed as automated transactions or be sent to a user as recommendations so that the user can decide whether to proceed with the transaction determined by the computer network 322.

Generally, while the recommendations to the user can be through any suitable electronic means, typically, the recommendations will be sent to an electronic display for review by the user. The user can access the electronic display through a dedicated computer system or through a website. The electronic display can be configured to provide a dashboard style user interface for the user to monitor the data (attributes and calculated values) and recommendations in real-time. Additionally, the electronic display can provide the customer with flow rate data, an estimation of the time at which a tank will reach capacity, and alerts should the tank level fall.

As discussed, for each tank, the computer network 322 will typically either recommend selling all or a portion of the contents of each tank as it currently exists (step 204), mixing and homogenizing the contents of each tank (step 206), removing and treating all or a portion of the contents of each tank (step 208) or wait and allow one or more of the tanks to continue to receive petroleum (step 210). The computer network 322 makes such determinations in light of the likely price to be received for the contents of each tank as well as the anticipated costs of various options. The computer network 322 will recommend the action to a user or take the action that produces the most profit for the owner of the petroleum. Additionally, the system makes recommendations for how the petroleum inside each tank should be treated by considering other tanks in the same or other fields. The computer network 322 can make recommendations including those recommendations discussed above.

In some embodiments, computer network 322 will be connected to agitators 354a-c and to valves (not shown) controlling the flow through petroleum outputs 332a-c, and conduits 340, 342, 362 and 364, and to control the operation of treatment unit 360. The connections are not shown in the drawings but will be understood by one skilled in the art based on the above disclosure. By these connections, the selling of the petroleum can be automated with computer network 322 disposing of the petroleum as indicated by the determination of an advantageous disposal, which typically will be the most profitable disposal of the petroleum. For example, these connections allow computer network 322 to direct petroleum outputs 332a-c to introduce petroleum to the system output through conduit 340 upon the determination that the advantageous disposal corresponds to a first set of conditions, or to direct petroleum outputs 332a-c to introduce petroleum to treatment unit 360 and activating treatment unit 360 upon the determination that the advantageous disposal corresponds to a second set of conditions. Further, computer network 322 can maintain all the petroleum in tanks 314a-c upon the determination that the advantageous disposal corresponds to a third set of conditions. Additionally, computer network 322 can activate the agitators 354a-c, prior to introducing the petroleum to the system output through conduit 340, upon the determination that the advantageous disposal corresponds to a fourth set of conditions.

Other embodiments will be apparent to those skilled in the art from a consideration of this specification or practice of the embodiments disclosed herein. Thus, the foregoing specification is considered merely exemplary with the true scope thereof being defined by the following claims.

What is claimed is:

1. A system for determining quality of petroleum:
a tank having a height, the tank containing the petroleum;
a plurality of sensors spaced apart along the height of the tank such that there is a sensor height associated with each sensor, wherein each sensor is configured to measure one or more attributes of a portion of the petroleum located at the associated sensor height;
a computer configured to receive the measured attributes from the sensors and calculate the quality of the portion of the petroleum at each sensor height.

2. The system of claim 1, wherein the attributes of the petroleum include a dielectric constant, conductivity, temperature and pressure.

3. The system of claim 2, wherein the computer calculates one or more calculated values from the attributes and determines the quality of the petroleum from the calculated values.

4. The system of claim 3, wherein the calculated values of the petroleum include suspended water content, salt content, BS&W content, API gravity, and free water content.

5. The system of claim 1, further comprising:
a transmission unit, wherein the transmission unit is in electronic communication with the sensors and the computer.

6. The system of claim 6, further comprising:
a petroleum output associated with the tank such that petroleum can be removed from the tank;
a treatment unit in fluid flow communication with the petroleum output such that the treatment unit can be activated to receive petroleum from the tank and treat the petroleum to remove one or more components from the petroleum to produce a treated petroleum; and
a system output in fluid flow communication with the petroleum output and the treatment unit such that at least a first portion of the petroleum removed from the tank can be removed from the system without treatment and such that at least a portion of the treated petroleum can be removed from the system.

7. The system of claim 6, wherein the computer is configured to determine an advantageous disposal of the petroleum based on the quality of the petroleum at each sensor height and wherein the advantageous disposal includes selling the first portion of the petroleum removed from the tank upon a first set of conditions, selling the portion of the treated petroleum upon a second set of conditions, and selling no portion of the petroleum upon a third set of conditions.

8. The system of claim 7, wherein the computer is configured to:
direct the petroleum output to introduce the first portion of petroleum to the system output upon the first set of conditions; and
direct the petroleum output to introduce some or all of the petroleum removed from the tank to the treatment unit and activating the treatment unit upon the second set of conditions.

9. The system of claim 8, further comprising an agitator associated with the tank such that when activated the agitator blends the petroleum in the tank to produce a homogenated petroleum.

10. The system of claim 9, wherein in the computer is further configured to activate the agitator upon a fourth set of conditions.

11. The system of claim 1, wherein:
the attributes of the petroleum include a dielectric constant, conductivity, temperature and pressure;
the computer calculates one or more calculated values from the attributes and determines the quality of the petroleum from the calculated values,
the calculated values of the petroleum include suspended water content, salt content, BS&W content, API gravity, and free water content.

12. A method for determining the quality of petroleum in a tank, the method comprising:
(a) measuring one or more attributes of the petroleum, wherein the attributes are measured at a plurality of heights within the tank such that a set of attributes is measured for each height, and the set of attributes reflect the properties of the petroleum at the height at which the set was taken;
(b) transmitting each set to a computer;
(c) calculating from each set one or more calculated values, which reflect the quality of the petroleum at the corresponding height;
(d) determining an advantageous disposal of the petroleum based on the quality of the petroleum at each height; and
(e) processing the petroleum based on the advantageous disposal.

13. The method of claim 12, wherein the attributes of the petroleum include a dielectric constant, conductivity, temperature and pressure.

14. The method of claim 12, wherein the calculated values include suspended water content, salt content, BS&W content, API gravity and free water content.

15. The method of claim 12, wherein the advantageous disposal includes the options of selling an untreated portion of the petroleum upon a first set of conditions; selling a treated portion of the petroleum upon a second set of conditions; and selling no portion of the petroleum upon a third set of conditions, and wherein the treated portion has been treated to remove one or more components from a portion of petroleum withdrawn from the tank, and the untreated portion of petroleum has no components removed from the portion of petroleum withdrawn from the tank.

16. The method of claim 15, wherein the step of determining an advantageous disposal includes determining the profit associated with options.

17. The method of claim 16, wherein the advantageous disposal further includes the option of blending the petroleum in the tank to create a homogenous petroleum prior to selling the untreated portion and the option of not blending the petroleum in the tank prior to selling the untreated portion.

18. The method of claim 17, wherein the advantageous disposal further includes the option of blending the petroleum in the tank to create the homogenous petroleum prior to treating the portion of petroleum withdrawn from the tank, and the option of not blending the petroleum in the tank prior to treating the portion of petroleum withdrawn from the tank.

19. The method of claim 18, wherein the attributes of the petroleum include a dielectric constant, conductivity, temperature and pressure.

20. The method of claim 19, wherein the calculated values include suspended water content, salt content, BS&W content, API gravity and free water content.
